(19) United States
(12) Patent Application Publication
(10) Pub. No.: US 2023/0382879 A1
(43) Pub. Date: Nov. 30, 2023

(54) Production process of alkylene oxides from alkylene carbonates

(71) Applicant: Politecnico di Milano, Milano MI (IT)

(72) Inventors: Davide MOSCATELLI, Arese MI (IT); Mattia SPONCHIONI, Binasco MI (IT); Alberto CESANA, Verano Brianza MB (IT); Gianmarco POLOTTI, Sesto San Giovanni MI (IT); Loïc RONIN, Milano (IT)

(21) Appl. No.: 18/245,909

(22) PCT Filed: Sep. 13, 2021
(86) PCT No.: PCT/IB2021/058294

§ 371 Date: Mar. 20, 2023

(30) Foreign Application Priority Data
Sep. 24, 2020 (IT) 102020000022624

(51) Int. Cl. C07D 303/04

(52) U.S. Cl. CPC C07D 303/04

(57) ABSTRACT

Catalytic process for producing alkylene epoxide, selected between ethylene oxide or propylene oxide, from the corresponding alkylene carbonate, selected between ethylene carbonate or propylene carbonate, comprising the decomposition reaction of alkylene carbonate, in the presence of sodium bromide as catalyst,

in which:
the reaction temperature is between 207 and 245° C., and
the catalyst is in amounts comprised between 5×10⁻⁴ and 8×10⁻³ moles per mole of alkylene carbonate.

This process can be carried out continuously. A further object of the invention is the modular plant which allows carrying out such a process.

### FIELD OF THE INVENTION

The present invention relates to a catalytic process for producing alkylene epoxides from the corresponding alkylene carbonates, which can be conducted continuously and the relative modular plant which exploits the advantageous operating conditions of such a process.

### BACKGROUND ART

Ethylene oxide and propylene oxide are chemical compounds predominantly used as reaction intermediates for industrial purposes in the production of numerous other chemical compounds. These include the industrial production of plastics and detergents in addition to the use thereof in the petrochemical and agrochemical field. The reactivity thereof is attributable to the high ring strain of the molecule. Furthermore, ethylene oxide can kill bacteria, moulds and fungi; as such, it is used, for example, as a sterilizing agent in medical devices and materials.

Despite the widespread use thereof, ethylene oxide and propylene oxide are volatile, extremely reactive, flammable and explosive gases. For example, the vapours thereof can give explosive decomposition, even in the absence of air (self-polymerization). Ethylene oxide is toxic by inhalation, irritating to the respiratory tract and potentially carcinogenic, especially in the liver. Propylene oxide is a suspected carcinogenic and mutagenic agent.

Like all hazardous substances, the transport thereof creates a constraint in the supply and logistics chain of the main industrial users (Guidelines for the distribution of ethylene oxide, CEFIC sector group, 4 ed, 2013, Guidelines for the distribution of propylene oxide, CEFIC sector group, 34 ed, 2019). In particular, the transport of these oxides must be subject to ADR (European agreement concerning the international carriage of dangerous goods by road). For these reasons, carriers implement several risk reduction plans with the careful choice of routes and logistics.

Reactive and gaseous ethylene oxide and propylene oxide are then usually stored in tanks or cisterns; thereby, however, the risk of uncontrolled reactivity and explosions increases, especially when the reagents are stored in large amounts and are not directly used for the specific use. The safety radius for the explosion of an industrial ethylene oxide tank, for example, can be several hundred metres, with serious consequences for the population and the surrounding environment.

This type of problem is not new in the chemical field and several solutions are possible. The safest solution is to turn hazardous substances into non-toxic, stable precursors. In the case of ethylene and propylene oxide, the two corresponding precursors are ethylene carbonate (CAS 96-49-1) and propylene carbonate (CAS 108-32-7). These substances, respectively solid and liquid, have reassuring toxicity profiles and do not cause transport and storage problems.

The chemical decomposition reaction of alkylene carbonate, as a precursor of alkyloxides, which occurs according to the following scheme:

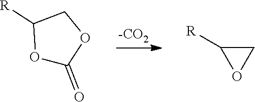

wherein R═H or lower alkyl, provides the use of catalysts.

Possible secondary reactions occur due to the reactivity of the alkylene epoxides, with themselves in the formation of polyglycols which are deposited on the bottom of the reactor. Literature data also suggest secondary rearrangement reactions of the alkylene epoxides themselves to give rise to aldehydes and acids.

U.S. Pat. No. 2,851,469 uses as catalysts a number of polyhalogenated hydrocarbons such as hexachloro methane, decachlorobutane, and pentachlorobutane, with a preference for hexachlorocyclohexane for the decomposition reaction of alkylene carbonates to epoxy alkylene.

U.S. Pat. No. 4,851,555 reports use as catalysts for such reaction halogenated arsonium salts such as tetrabutylarsonium iodide, triphenyl methyl arsonium iodide, tetraphenyl arsonium chloride, bromide or iodide.

EP0047473 includes the use of alkali bromides, less toxic than arsonium salts and, with respect to the processes described, has higher conversions as the Applicant has experimentally verified and as reported in the following table 1.

TABLE 1

| | Conversion |
|---|---|
| Catalyst | % (4 hours |
| Hexachlorocyclohexane | about 20 |
| Tetraphenyl arsonium chloride | about 60₁₀ |
| Sodium bromide | about 70 |

Although sodium bromide is decidedly more efficient and less toxic as a catalyst, the decomposition rate is still rather low while in the meantime the epoxide formed, which remains trapped in the liquid phase, converts into by-products itself. To overcome this problem, the reaction is conducted at pressures much lower than atmospheric pressure.

The content of EP0047474 is similar to that of EP0047473; in this case, again in order to minimize the formation of undesirable products, an inert gas flow is used within the liquid phase to at least partially remove the epoxide product formed. This inert gas can be CO₂, nitrogen or helium.

U.S. Pat. No. 4,192,810 describes a mode for preparing vicinal epoxides, but does not include the use of NaBr as a catalyst.

The article “Decomposition of ethylcarbonate in the presence of alkali metal halides”, Shapiro et al, ZHURNAL ORGANICHESKOI KHIMII, vol. 5, 1969, pages 207-212 discloses a process for the decomposition of ethylene carbonate in the presence of an alkali metal halide, such as NaBr, at different temperature ranges and catalyst amounts from those of the present invention as set forth below in the present disclosure.

### SUMMARY OF THE INVENTION

The applicant has developed a catalytic process for producing alkylene oxide, selected between ethylene oxide or propylene oxide, from the corresponding alkylene carbonate, selected between ethylene carbonate or propylene carbonate, according to the following scheme:

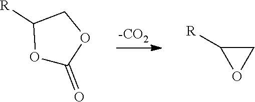

wherein R═H or methyl, and using sodium bromide as catalyst in which:
   the reaction temperature ranges between 207 and 245° C.,
   the catalyst is in amounts comprised between 5×10⁻⁴ and 8×10⁻³ moles per mole of alkylene carbonate.

The catalytic process object of the invention is advantageous with respect to the known processes, and in particular with respect to that disclosed in EP0047473, because it allows to obtain alkylene oxide from alkylene carbonate, using a lower amount of catalyst, avoiding operating under vacuum.

Furthermore, reaction products, remaining trapped in the liquid phase, are prevented from giving rise to unwanted by-products, as instead occurs in EP0047473, which in order to avoid this leads to the same reaction under reduced pressure.

Thanks to the aforementioned advantageous operating conditions with which the process is carried out, it has been possible to develop a modular plant which is easy to build, which can also be of limited size, thus reducing the risk of explosions, which occur more frequently in the phase of storage and transport of large amounts of alkylene epoxides.

This plant, thanks to the fact that it can be limited in size and is modular, can, for example, be easily installed on site where it is necessary to have daily amounts of alkylene epoxides, for example in small industrial companies or in laboratories.

The on-site generation of alkylene epoxide and the possible and subsequent direct use thereof in further reactions to give products for other industrial uses solve the problems related to the transport and storage of said material.

### DESCRIPTION OF THE FIGURES

FIG. 1 depicts preferred embodiments of the modular plant in which the alkylene epoxide is produced according to the process of the invention.

FIG. 2 shows the embodiments of the reactor of the module of the modular plant in which the alkylene epoxide is produced according to the process of the invention.

FIG. 3 shows the temperature variation as a function of the reaction time observed for tests 1-6 of example 1 using specific catalyst/ethylene carbonate ratios.

FIG. 4 shows the temperature variation as a function of the reaction time observed for tests 7-13 of example 1 using specific catalyst/propylene carbonate ratios.

FIG. 5 graphically depicts the values given in table 2 of example 1 of the decomposition time as a function of the different molar ratios of sodium bromide/ethylene carbonate used in tests 1-6 of the same example.

FIG. 6 graphically depicts the values given in table 2 of example 1 of the decomposition time as a function of the different molar ratios of sodium bromide/propylene carbonate used in tests 7-13 of the same example.

FIG. 7 shows the ¹H-NMR spectrum of the pentanol solvent/reagent before coming into contact with ethylene oxide

FIG. 8 shows the ¹H-NMR spectrum of the reaction product between ethylene oxide with pentanol to give pentanol ethoxylate.

### DETAILED DESCRIPTION OF THE INVENTION

For the purposes of the present invention, the verb “comprising” is intended to define a set of elements, expressly indicating some, without excluding the presence of others not expressly indicated; while the term “constituting” or “constituted” is intended to define a set of elements, expressly indicating them all, and thus excluding the presence of components not expressly listed.

In the process of the invention when the reagent is ethylene carbonate, the reaction temperature is preferably comprised between 221 and 235° C. and said catalyst is present in amounts preferably comprised between 7×10⁻⁴ and 5×10⁻³ moles of catalyst/ moles of ethylene carbonate.

In the process of the invention, when the reagent is propylene carbonate the reaction temperature is preferably comprised between 237 and 243° C., the mole ratio of catalyst/propylene carbonate is preferably comprised between 8×10⁻⁴ and 7×10⁻³.

As noted above, the process according to the present invention operates at pressures close to atmospheric pressure; therefore, it is not necessary to operate at pressures lower than atmospheric pressure as is the case with EP0047473.

In particular, the decomposition reaction of the alkylene carbonate of the catalytic process preferably occurs at a reaction pressure comprised between 1 and 50 bar, more preferably at a pressure comprised between 1 and 10 bar.

As illustrated above, a further object of the invention is the modular plant of relatively limited size which allows the catalytic process to be carried out in accordance with the present invention.

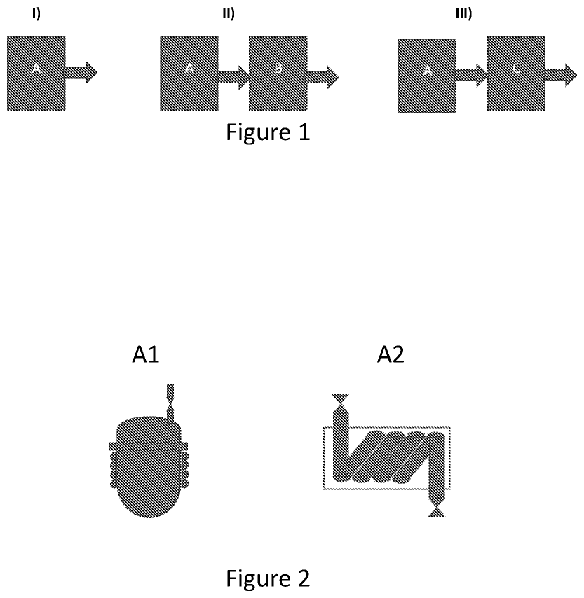

This modular plant can consist of a single module (A) comprising the reactor in which the formation of the alkylene oxide occurs according to the process of the invention, i.e., the alternative I) shown in FIG. 1.

This module can possibly be associated with a module (B) comprising a CO₂ blast chiller, or alternative II) shown in FIG. 1, or can be associated with a module (C) according to alternative III) shown in FIG. 1, comprising a reactor in which the alkylene epoxide, which exits from the module (A), is subjected to a further reaction in the presence of a suitable reagent to give a product for industrial use.

According to a further alternative not shown in the figure, the plant according to the present invention contains all three modules (A), (B) and (C),

The module (A) is associated or connected with the module (B) or (C) by means of hydraulic/mechanical connection means, such as valve/flange/valve.

The reactor of module (A) preferably consists of material capable of (or suitable for) resisting the pressures of the gases formed at the process temperature and pressure. Preferably, the catalytic reactor (A) is made of steel, more preferably it is made of stainless steel 316—but it can also be made of glass or ceramic material, in the latter two cases it can be further enclosed in a metal or plastic protection. A specific system of pressure regulating valves ensures the set point pressure (or preset pressure) and the possible vent in the case of overpressure.

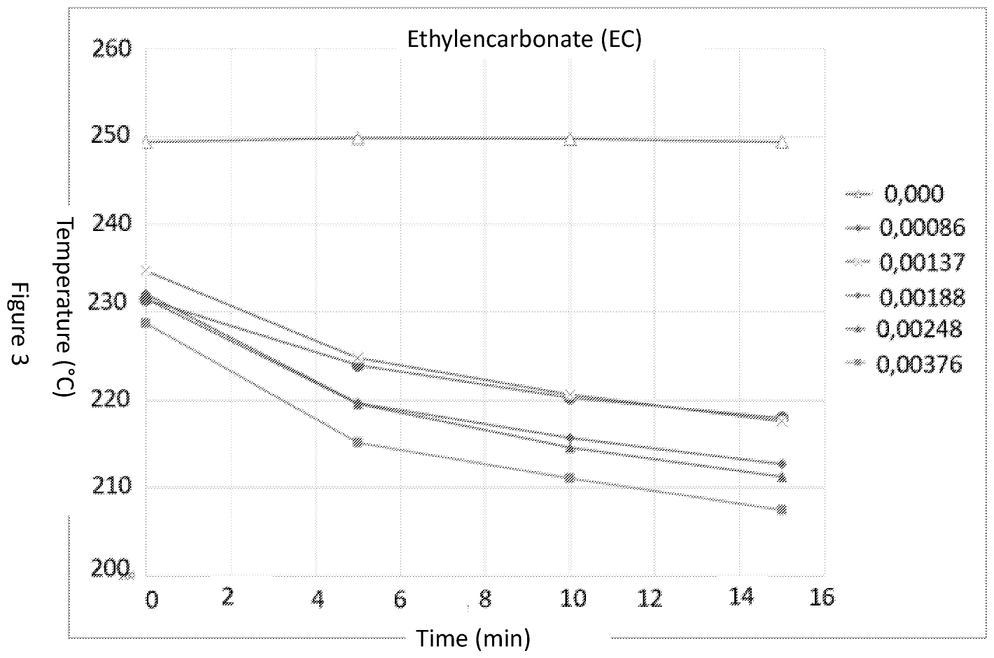

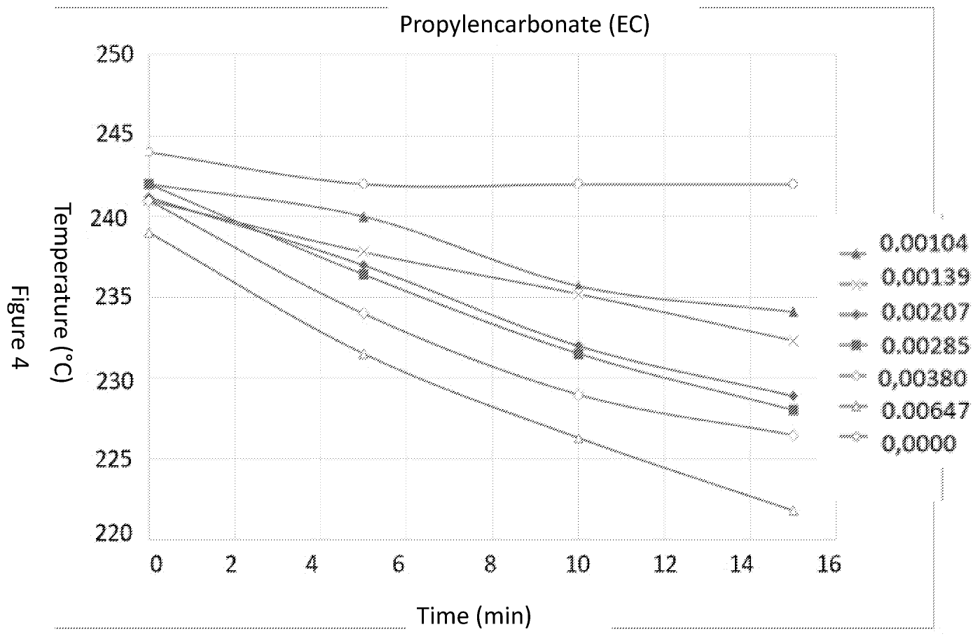

According to a preferred embodiment of the invention, the module (A) can comprise at least one batch reactor indicated as (A1) in FIG. 2 of cylindrical shape possibly provided with a stirrer, externally thermostated through electrical resistors or through a contact fluid (oil or steam) or through irradiation (IR lamps).

The process can be conducted continuously or discontinuously, preferably it is conducted continuously.

When the process of the invention is conducted continuously, the module (A) can comprise a single batch reactor (A1) also defined as CSTR (Continuous Stirred Tank Reactor) or multiple CSTRs (A1) arranged in series or in parallel with each other. This solution is preferred in the case where alkylene epoxide is to be produced in mass quantities.

The size of the batch reactor for the discontinuous process or for the CSTR is preferably comprised between 1 cm³ and 10 m³, more preferably between 1 dm³ and 1 m³.

According to another preferred embodiment, to conduct the process continuously the reactor of the module (A) has a tubular shape (A2) of the PFR (Plug Flow Reactor) type as shown in FIG. 2, where it specifically has a coiled shape and is immersed in a heating fluid bath, indicated in the figure with the rectangle.

According to another preferred embodiment, the tubular reactor is inserted inside a tubular sleeve arranged for the entire length of the reactor and in which a heating fluid passes.

The module (A), in addition to the reactor, will comprise support units such as steam supply units for heating, units for cooling the exhaust gases, units for melting the alkylene carbonate, pumping and reagent supply units in the reactor.

The module (A) can further comprise devices for feeding inert gases in liquids to facilitate the removal of gaseous products (alkylene oxides and CO₂). Preferably, said inert gases are selected between: nitrogen, and helium.

The module (A) will comprise different forms of reactor instrumentation and control such as meters and specifically pressure, temperature, conductivity, heat, viscosity, infrared spectrum sensors at both medium and low frequencies. In the plant in which the process of the invention is carried out continuously, particular care will be taken to control the flow rate of the input liquid, while in any case, it will be important to measure the total output gaseous flow rate. Thus the module (A) will comprise mass or volume flow meters.

All this instrumentation will be coordinated according to the usual control techniques, by means of one or more central units or the so-called programmable logic controllers (PLC) attached to the module or relocated to a remote location (distributed control systems, DCS).

The reactor of module (A) advantageously allows to reduce or even cancel the amount of inert gas necessary to ensure a continuous flow and thus allow the passage of the gaseous products to the other operating units.

The module (A) preferably comprises, downstream of the reactor, a condenser or a heat exchanger which allows the separation of the unreacted alkylene carbonate, which is separated and recycled from the reaction products in gaseous form, such as CO₂ and alkylene epoxide, which are conveyed to module (B) or (C). The condenser or heat exchanger is a tube bundle with mains water inside for cooling. The condenser or heat exchanger can comprise instrumentation units for measuring, controlling and stabilizing the internal temperature and pressure. The temperature and pressure conditions of the condenser (or heat exchanger) are such that the unreacted alkylene carbonate in condensed form can return to the reaction mixture to undergo decomposition; at the same time, the reaction products, CO₂ and alkylene epoxide undergo cooling but remain in the form of non-condensable gases, and can be conveyed to the module (B) comprising the CO₂ abatement unit to separate the alkylene epoxide from the CO₂, to be used as a disinfectant.

The CO₂ blast chiller of module (B) is preferably a filler or perforated-plate adsorption column.

As noted above, the reaction mixture output from the module (A) is preferably conveyed to the module (C).

In this case, the alkylene epoxide and the CO₂, after having undergone a cooling process by means of the condenser, are sent to the module reactor (C) in which the alkylene epoxide undergoes a further reaction and the CO₂ used as an inert gas.

The reactions which can occur in the reactor of module (C) are for example:
fatty alcohol alkyleneoxylation reaction, such as lauryl alcohol, coconut or palm alcohol, linear or branched petrochemical-derived alcohols;
alkyleneoxylation reaction of acrylate compounds selected from: acrylic acid or methacrylic acid;
alkyleneoxylation reaction of diols;
reaction with hydrochloric acid or hydrogen sulfide for the production of 2-chloroethanol, 2-chloropropanol, 2 hydroxychloropropane, hydroxythioethane, 2-hydroxythiopropane, 2-thiopropanol, reactions catalysed by magnesium chloride or other alkali metal or alkali earth salts
alkyleneoxylation reaction of polysaccharides or celluloses in flour or powder such as hemicellulose, carboxymethylcellulose, carboxyethylcellulose, guar gum, xanthan gum, alginates, amides.

The alkyleneoxylation reaction of acrylate compounds, selected between acrylic acid or methacrylic acid, allows to obtain ethoxylated monomers which can then be used in acrylate polymerization reactions.

Likewise, the alkyleneoxylation reaction of diols allows to obtain ethoxylated monomers which can then be used in urethane polymerization reactions.

Module (C) preferably comprises a reactor which is suitable for gas-liquid or gas-solid reactions.

The reactor of module (C) is suitable for conducting gas-liquid reactions and is preferably selected from flat or filler columns, coiled tubular reactors, heat exchangers.

When module (C) comprises a reactor suitable for gas-solid reactions, the reactor is preferably selected between a fluid bed reactor, extruder, powder compactor, granulator.

The Applicant gives examples merely for illustrative and non-limiting purposes of the invention below.

### Example 1— Experimental Tests of Ethylene Carbonate and Propylene Carbonate Decomposition

The decomposition reaction was studied by conducting the reaction in batches. The experimentation was carried out considering EC ethylene carbonate and PC propylene carbonate.

The decomposition reactions of both ethylene carbonate and propylene carbonate of the catalytic process are endothermic reactions with a measured value of reaction enthalpy (ΔH_{R} in kJ/mol), as measured by the enthalpy of reactant and product formation, equal to 219.09 kJ/mol for EC ethylene carbonate and equal to 124.81 kJ/mol for PC propylene carbonate. The Applicant carried out a series of experimental tests shown in table 2 for both ethylene carbonate and propylene carbonate, proceeding as follows:
(a) initial heating step of the reactor of module (A) where the thermal power supplied is between 90 and 100 W (sleeve of module (A) set to an instrument value of 6.8);
(b) secondary decomposition/production and heating step from the maximum temperature reached in the initial step (a) and for the following 15 minutes with a value of deliverable thermal power between 120 and 125 W (sleeve of module (A) set at an instrument value of 10.0).

Each test, for both EC ethylene carbonate and PC propylene carbonate, was performed with the same molar amount of alkylene carbonate (50 g and 100 g for EC; 58 g and 116 g for PC). The tests therefore differ only for the amount of catalyst (NaBr) added. Below are the results obtained in the same format as the previous series, for ease of reading.

6 tests were performed using ethylene carbonate and 7 tests using propylene carbonate.

The alkylene oxide obtained as a product was measured by titration; specifically, the alkylene oxide product was adsorbed with a catalysed acid (HCl) bath (MgCl₂) (measurement by titration of the amount of alkylene epoxide formed by reaction with MgCl₂ catalysed hydrochloric acid by the method proposed by F. W. Kerckow in Analytische Bestimmung von Äthylenoxydthylenoxyd, Analytical and Bioanalytical Chemistry, volume 108, issue 7-8, 1937). The hardware (glass fittings, etc.) of all the tests is identical.

During the decomposition reaction, a lively formation of gas and a progressive reduction in the volume of the liquid phase is observed.

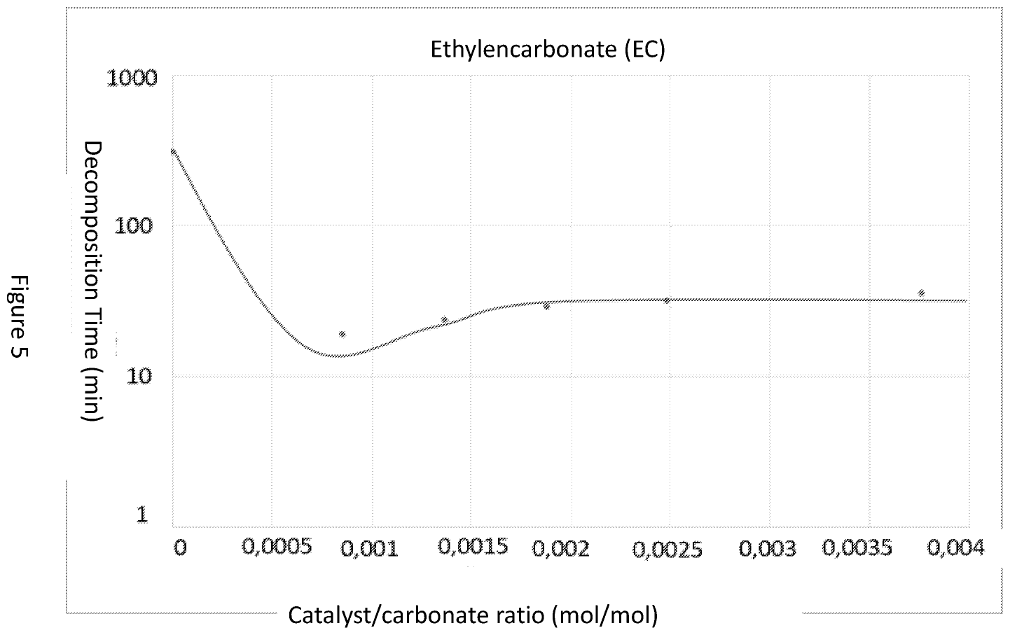

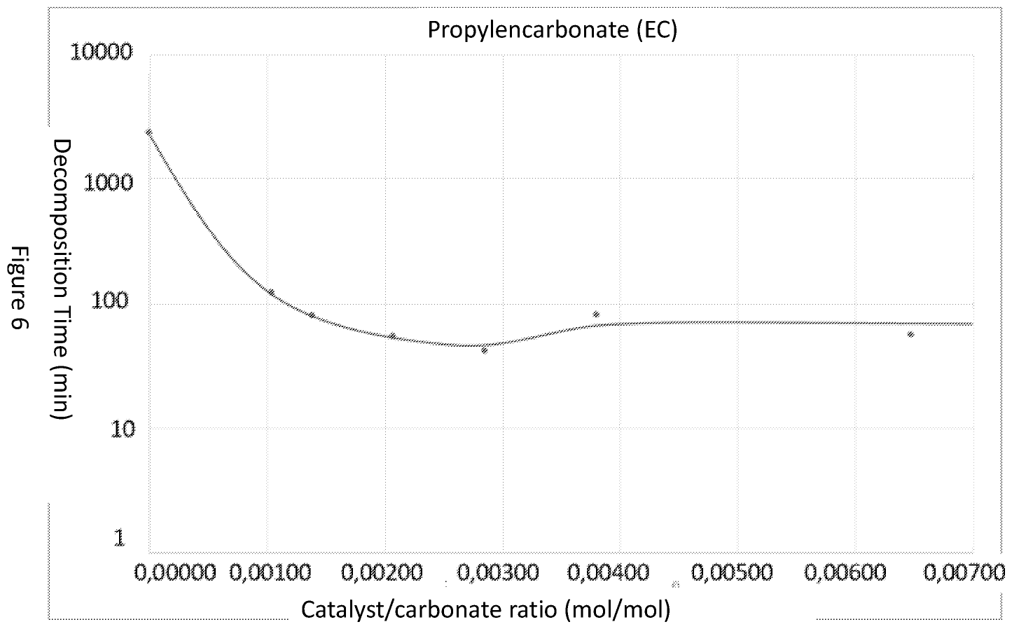

The graphs in FIGS. 4 and 5 show the change in temperature as a function of time for tests 1-6 wherein the reagent is ethylene carbonate and for tests 7-13 where the reagent is propylene carbonate, respectively.

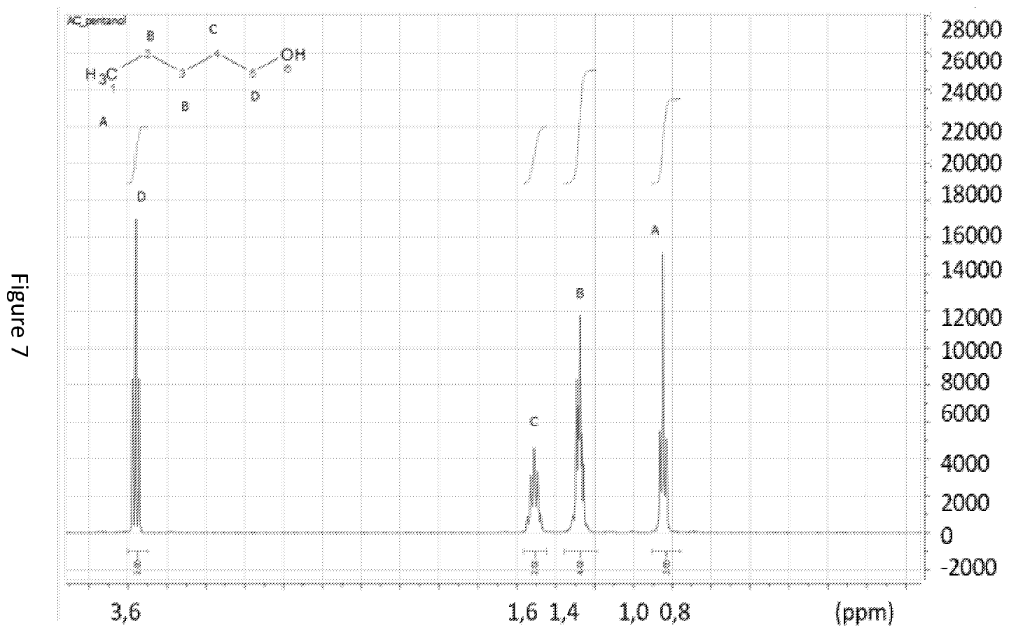

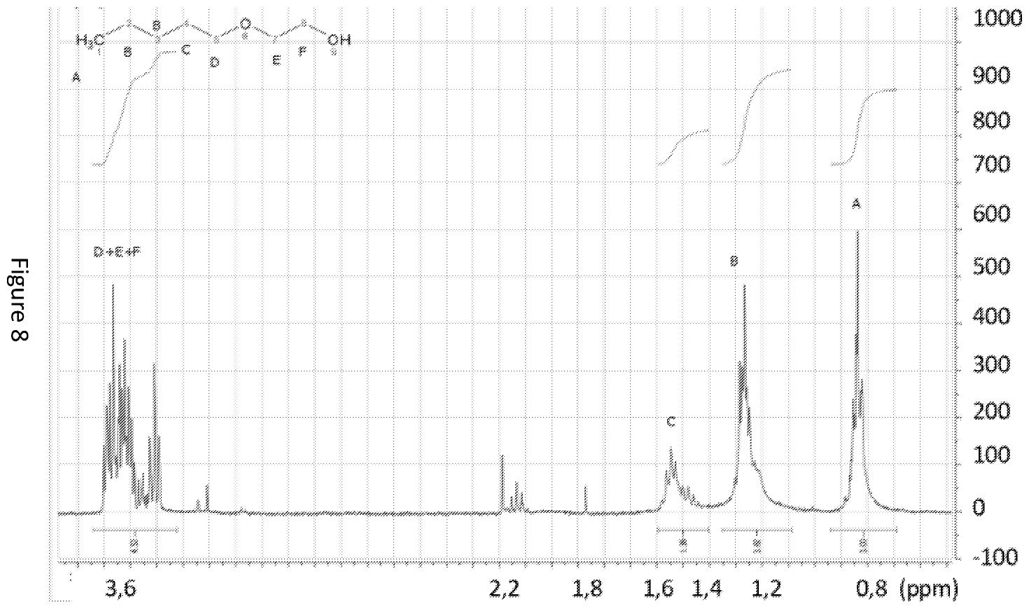

The Applicant reports below, in the aforesaid table 2 the decomposition time as a function of the ratio of catalyst moles/starting alkylene carbonate moles. The results obtained are also depicted in the graphs of FIGS. 5 and 6.

TABLE 2

| | | Formation time | | | Formation time |
|---|---|---|---|---|---|
| | Moles | of 0.50 mol of | | Moles | of 0.50 mol of |
| | NaBr/ | ethylene oxide | | NaBr/ | propylene oxide |
| | EC | from 100 g of | | PC | from 116 g of |
| Test | moles | EC (minutes) | Test | moles | PC (minutes) |
| 1 | 0.00000 | 310 | 7 | 0.00000 | 2350 |
| 2 | 0.00086 | 19 | 8 | 0.00104 | 122 |
| 3 | 0.00137 | 24 | 9 | 0.00139 | 80 |
| 4 | 0.00188 | 29 | 10 | 0.00207 | 55 |
| 5 | 0.00248 | 32 | 11 | 0.00285 | 42 |
| 6 | 0.00376 | 36 | 12 | 0.00380 | 82 |
| | | | 13 | 0.00647 | 56 |

The formation time of 0.50 mol of ethylene oxide from the initial 100 g of ethylene carbonate occurs in a minimum time of 19 minutes using 0.00086 moles of NaBr per mole of ethylene carbonate and under the experimental conditions described in terms of pressure and temperature. The decomposition reaction of ethylene carbonate in the absence of sodium bromide catalyst does not occur.

The formation time of 0.50 mol of propylene oxide from the initial 116 g of propylene carbonate occurs in a minimum time of 42 minutes using 0.00285 mol NaBr per mole of propylene carbonate and under the experimental conditions described in terms of pressure and temperature. In this case, the propylene carbonate decomposition reaction in the absence of sodium bromide catalyst is very slow.

### Example 2— Ethoxylation Reaction

The ethylene oxide generated in module (A) and conveyed after module (C) as described above can immediately react with an alcohol, ethoxylating it. The NMR analysis of the initial solvent/reagent (pentanol) is shown in the ¹H-NMR spectrum in FIG. 8; while FIG. 9 shows the transformation thereof into ethoxylated alcohol (left peaks increased in number and intensity), obtained by bubbling ethylene oxide at a temperature comprised between 180 and 200° C. and using sodium methylate in pentanol as catalyst.

1. Catalytic process for producing alkylene epoxide, selected between ethylene oxide or propylene oxide, from the corresponding alkylene carbonate, selected between ethylene carbonate or propylene carbonate, comprising the decomposition reaction of alkylene carbonate, in the presence of sodium bromide as catalyst, according to the following scheme: 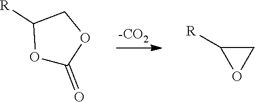
with R═H, methyl and
wherein:
the reaction temperature ranges between 207 and 245° C.,
the catalyst is in amounts comprised between 5×10⁻⁴ and 8×10⁻³ moles per mole of alkylene carbonate.

2. The process of claim 1 wherein, when the reagent is ethylene carbonate, the reaction temperature ranges between 221 and 235° C. and said catalyst is present in amounts comprised between 7×10⁻⁴ and 5×10⁻³ moles of catalyst/moles of ethylene carbonate.

3. The catalytic process of claim 1, wherein when the reagent is propylene carbonate the reaction temperature ranges between 237 and 243° C., the mole ratio of catalyst/propylene carbonate ranges between 8×10⁻⁴ and 7×10⁻³.

4. The catalytic process according to [[any one of claims from]] claim 1 wherein the pressure ranges between 1 and 50 bar, preferably between 1 and 10 bar.

5. The catalytic process according to claim 1, conducted in a modular plant comprising a module (A) in turn comprising the reactor in which said catalytic demolition of the alkylene carbonate occurs, and associated with:
a module (B) comprising a CO₂ blast chiller, or
a module (C), comprising a further reactor in which the alkylene oxide from the module (A) is subjected to a further reaction to give an industrial product.

6. The catalytic process according to claim 1, conducted in a modular plant comprising a module (A) in turn comprising the reactor in which said catalytic demolition of the alkylene carbonate occurs, and associated with:
a module (B) comprising a CO₂ blast chiller and
a module (C), comprising a further reactor in which the alkylene epoxide from the module (A) is subjected to a further reaction to give an industrial product.

7. The catalytic process according to claim, wherein the module (A) comprises at least one batch reactor of cylindrical shape (A1), possibly provided with a stirrer, externally thermostated through electrical resistors, through a contact fluid or through irradiation.

8. The process according to claim 1, conducted continuously or discontinuously, preferably continuously.

9. The catalytic process according to claim 8, wherein when said process is conducted continuously, the module (A) comprises a single CSTR reactor (A1), or multiple CSTR reactors (A1), said multiple reactors being arranged in series or in parallel with each other.

10. The catalytic process according to claim 5, wherein the module (A) is tubular in shape (A2) and is thermostated by immersion in a heating fluid bath or is inserted inside a sleeve arranged for the entire length of the reactor in which a heating fluid passes.

11. The catalytic process according to claim 5, wherein the module (A) comprises downstream of the reactor a condenser or an exchanger which allows the separation of the unreacted alkylene carbonate from the reaction products in gaseous form, the CO₂ and the alkylene epoxide, said unreacted alkylene carbonate being recycled in the reactor of the module (A), while the aforementioned reaction products in gaseous form, CO₂ and alkylene epoxide, are conveyed to the module (B) or (C).

12. The catalytic process according to claim 5, wherein the module (B) comprises a filler or perforated-plate adsorption column.

13. The catalytic process according to claim 5, wherein the alkylene epoxide and CO₂ formed in the reactor (A) are sent to the module (C) where they are subjected, in the presence of a specific reagent, to a further reaction, the CO₂ has the function of inert gas and said reactor is adapted to gas-liquid or gas-solid reactions.

14. The catalytic process according to claim 13, wherein said reactor of the module (C) is adapted to gas-liquid reactions and is selected from flat or filler columns, coiled tubular reactors, heat exchangers.

15. The catalytic process according to claim 14, wherein said reactor of the module (C) is adapted to gas-solid reactions and is selected from a fluid bed reactor, extruder, powder compactor, granulator.

16. The process according to claim 13, wherein said further reaction of the alkylene epoxide occurring in the reactor of the module (C) is selected from:
alkyleneoxylation reaction of fatty alcohols, such as lauryl alcohol, coconut or palm alcohol, linear or branched petrochemical-derived alcohols;
alkyleneoxylation reaction of acrylate compounds selected from: acrylic acid or methacrylic acid;
alkyleneoxylation reaction of diols;
reaction with hydrochloric acid or hydrogen sulfide for the production of 2-chloroethanol, 2-chloropropanol, 2 hydroxychloropropane, hydroxythioethane, 2-hydroxythiopropane, 2-thiopropanol, reactions catalysed by magnesium chloride or other alkali or alkaline earth metals salts
alkyleneoxylation reaction of polysaccharides or celluloses in flour or powder such as hemicellulose, carboxymethylcellulose, carboxyethylcellulose, guar gum, xanthan gum, alginates, amides.
